(19) United States
(12) Patent Application Publication
(10) Pub. No.: US 2025/0001081 A1
(43) Pub. Date: Jan. 2, 2025

(54) Sub-assembly for a medicament delivery device

(71) Applicant: SHL Medical AG, Zug (CH)

(72) Inventors: Tzu Wei Liu, Taoyuan City (TW); Meng-Jhen Chiou, Taoyuan City (TW)

(21) Appl. No.: 18/708,029

(22) PCT Filed: Nov. 2, 2022
(86) PCT No.: PCT/EP2022/080511

§ 371 Date: May 7, 2024

(30) Foreign Application Priority Data
Nov. 10, 2021 (EP) 21207550.1

(51) Int. Cl. A61M 5/20; A61M 5/32

(52) U.S. Cl. CPC A61M 5/20; A61M 5/3202; A61M 2207/10

(57) ABSTRACT

The present disclosure provides a sub-assembly for a medicament delivery device configured to expel medicament from a medicament container. The sub-assembly comprises a housing and a medicament container carrier. The medicament container carrier comprises a carrier wall having an open proximal end, at least one slot extending from the open proximal end in a distal direction, and at least a first leg portion and a second leg portion radially separated by the slot at least at the open proximal end such that the first and second leg portions are radially movable relative to each other. The sub-assembly further comprising a tubularly shaped retaining element and a cap comprising a protrusion extending distally into the proximal end of the housing, wherein the retaining element comprises a first engaging structure releasably engageable with an associated second engaging structure of the distally extending protrusion, such that the retaining element is releasably engaged to the distally extending protrusion.

### CROSS-REFERENCE TO RELATED APPLICATIONS

The present application is a U.S. National Phase Application pursuant to 35 U.S.C. § 371 of International Application No. PCT/EP2022/080511 filed Nov. 2, 2022, which claims priority to European Patent Application No. 211207550.1 filed Nov. 10, 2021. The entire disclosure contents of these applications are herewith incorporated by reference into the present application.

### TECHNICAL FIELD

The present disclosure generally relates to a medicament delivery device for expelling medicament from a medicament container. In particular, the present disclosure relates to a sub-assembly for a medicament delivery device, and to a method of assembly of a medicament delivery device comprising a sub-assembly. The present disclosure further relates to a kit of parts comprising an external fixture and a sub-assembly of a medicament delivery device and corresponding method of assembly of a medicament delivery device using the kit of parts.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a needle or a nozzle.

These days, a number of different medicament delivery devices exist, including various types of injectors (for example pen injectors, autoinjectors, on-body devices). Although many of these devices have enabled major improvements in the management of a number of medical conditions, various limitations do still exist in the current technology. Not least amongst these are the unintentional and undesired movement of the medicament container within the housing. In considering these problems, the applicant has appreciated that various developments could be made to help improve the medicament delivery devices on the market today, which are set out in more detail below.

### SUMMARY

An object of the present disclosure is thus to provide a sub-assembly for a medicament delivery device, and a medicament delivery device, which solves, or at least mitigates, problems of the prior art.

There is hence according to a first aspect of the present disclosure provided a sub-assembly for a medicament delivery device configured to expel medicament from a medicament container. The sub-assembly comprises: a housing having a proximal end and a distal end; and a medicament container carrier extending along a longitudinal axis from a proximal end to a distal end. The medicament container carrier comprises: a carrier wall having an open proximal end, at least one slot extending from the open proximal end in a distal direction, and at least a first leg portion and a second leg portion radially separated by the slot at least at the open proximal end such that the first and second leg portions are radially movable relative to each other. The sub-assembly further comprises a tubularly shaped retaining element extending along a longitudinal axis from a proximal end to a distal end, the retaining element comprising a proximal end portion extending distally from the proximal end, a distal end portion extending proximally from the distal end, and an inner surface adjacent to the first and second leg portions to radially support the first and second leg portions. The sub-assembly further comprises a cap arranged at least partly proximal the housing, the cap comprising a protrusion extending distally into the proximal end of the housing. The distal end portion of the retaining element comprises a first locking structure lockable with an associated second locking structure extending from at least one of the first and second leg portions of the carrier wall, such that the retaining element is locked to the open proximal end of the carrier wall by means of the first and second locking structures, and wherein the proximal end portion of the retaining element comprises a first engaging structure releasably engageable with an associated second engaging structure of the distally extending protrusion, such that the retaining element is releasably engaged to the distally extending protrusion.

An effect obtainable thereby is an improved radial retainment of the first and second leg portions in the housing. Hereby, a medicament container housed in the medicament container carrier will be prevented from undesired dislocation, e.g. by being prevented from at least partly slipping out proximally of the medicament container carrier. That is, in an assembled state of the medicament delivery device in which the medicament container carrier is arranged in a final proximal position within the housing, the inner surface of the tubularly shaped retaining element obstructs a radially outwardly movement of the first and second leg portions and thus prevent the first and second leg portions from moving radially outwards. Thus, the tubularly shaped retaining element retains, or keeps, the first and second leg portion in radial position to secure a medicament container within the medicament container carrier. Moreover, by the first and second locking structures, and the first and second engaging structures, the retaining element is efficiently arranged in its correct position within the housing to radially support the first and second leg portions.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to one embodiment, the housing is tubular or substantially tubular. Thus, an inner surface of the housing is typically an inner tubular or inner cylindrical surface, or may be referred to as an inner enveloping cylindrically shaped surface.

According to one embodiment, the medicament container carrier is tubular or substantially tubular. Thus, the carrier wall of the medicament container carrier is typically cylindrically shaped. The medicament container carrier is configured to fit inside the housing.

The tubularly shaped retaining element may comprise portions not being tubularly shaped, such as e.g. proximally or distally extending protrusions. The tubularly shaped retaining element is sometimes simply referred to as a retaining element.

Thus, the housing and/or the medicament container carrier, and/or the retaining element may be defined by cylindrical coordinates in a cylindrical coordinate system. Thus, a radial cross section of the housing, the medicament container carrier or the retaining element may be defined by polar coordinates in a polar coordinate system. A cylindrical structure advantageously provides for rotational symmetry.

According to one embodiment, the sub-assembly is configured to move from a pre-activation preventing state in which the retaining element is releasably engaged to the distally extending protrusion, into an activation admittible state in which the cap is removed and the distally extending protrusion is released from the retaining element by the release of the first engaging structure from the second engaging structure. Thus, the retaining element may be kept in position even after the cap has been removed and the sub-assembly is arranged in its activation admittible state. In the pre-activation preventing state, the protrusion extending distally into the proximal end of the housing typically prevent activation of the sub-assembly, e.g. by preventing axial and/or rotational movement of a components in the medicament delivery device. As the cap is removed, the distally extending protrusion is withdrawn from the proximal end of the housing and the activation prevention is annulled (e.g. by allowing axial and/or rotational movement of a components in the medicament delivery device).

According to one embodiment, the distally extending protrusion is a shield remover, such as an RNS remover, as it may be configured to remove a shield or RNS from the medicament container upon cap removal. The distally extending protrusion may be a tubular protrusion.

According to one embodiment, the first and second locking structures are configured as a snap-lock. A snap-lock is advantageous, as it may be used in a straightforward manner during assembly of the sub-assembly. A snap-lock may provide a relatively strong locking function. The snap-lock may according to one embodiment be configured irreversible locking. That is, according to one embodiment, subsequent to the snap-lock of the first and second locking structures, the snap-lock cannot be released without breaking the first and/or the second locking structures.

According to one embodiment, the snap-lock comprises a recess and a protrusion configured to be snap-locked into the recess, wherein the first locking structure comprise the recess or the protrusion, and the second locking structure comprises the other one of the recess and the protrusion. Thus, the first locking structure may comprise the recess, and the second locking structure comprise the protrusion, or the first locking structure may comprise the protrusion, and the second locking structure comprise the recess.

According to one embodiment, the first engaging structure comprises at least one flexible arm having a hooked-shaped end, and the second engaging structure comprises a protrusion configured to engage with the hook-shaped end of the flexible arm in a releasably engaging manner. Such releasably engaging means is advantageous as it may be used in a straightforward manner during assembly of the sub-assembly. Such releasably engaging means may provide a relatively weak engaging or locking function. The releasably engaging means is thus configured for reversible locking. That is, subsequent to engagement of the first and second engaging structure, the releasably engaging means can be released without breaking the first and/or the second engaging structures.

According to one embodiment, the protrusion of the second engaging structure is a circumferential protrusion. A circumferential protrusion advantageously provides for rotational symmetry. Thus, the circumferential orientation of the first engaging structure, such as the flexible arm with the hooked-shaped end is of less or no importance for causing a the releasably engagement.

According to one embodiment, the first engaging structure comprises a plurality of flexible arms arranged around the periphery of the retaining element, each one of the flexible arms having a hooked-shaped end engageable with the protrusion of the second engaging structure. Hereby, the releasably engagement of the first and second engaging structures may be improved. Moreover, the circumferential orientation of the second engaging structure, such as the protrusion, is of less or no importance for causing a the releasably engagement. Furthermore, the strength of the releasably engagement of the first and second engaging structures may be adapted by adapting the number of flexible arms.

According to one embodiment, the locking between the retaining element and the open proximal end of the carrier wall by means of the first and second locking structures, are stronger than the releasable engagement of the retaining element and the distally extending protrusion of the cap by means of the first and second engaging structures. Hereby, the sub-assembly may be brought into the activation admittable state as the cap is removed and the distally extending protrusion is released from the retaining element by the release of the first engaging structure from the second engaging structure, without dislocating the retaining element from its installed position. In other words, the retaining element may be locked into its correct position within the housing by means of the cap as the retaining element is releasably engaged to the distally extending protrusion of the cap, while still enabling the cap and the distally extending protrusion to be released from the retaining element subsequent to locking the retaining element in its correct position within the housing by locking to the medicament container carrier by means of the first and second locking structures.

According to one embodiment, the first and second engaging structures are configured to be released from each other upon an applied threshold force to the cap in the proximal direction, wherein the first and second locking structures are configured to remained locked to each other upon the application of the threshold force to the cap in the proximal direction. Hereby, the sub-assembly may be brought into the activation admittable state as the cap may be removed and the distally extending protrusion released from the retaining element by the release of the first engaging structure from the second engaging structure, without unlocking the retaining element from the medicament container carrier as the first and second locking structures are remained locked. For example, for embodiments in which the first and second locking structures are configured as snap-lock (e.g. by a protrusion and a recess) and wherein the first and second engaging structures comprises at least one flexible arm having a hooked-shaped end and a protrusion, respectively, to provide a releasably engaging means, the snap-lock is made stronger than the releasably engaging means. This may e.g. be achieved by adequate sizing and dimensioning of the recess and protrusion of the snap-lock, as well as adequate sizing and dimensioning of the hooked-shaped end of the at least one flexible arm and the corresponding protrusion.

According to one embodiment, the sub-assembly is arrangeable in a disassembled state in which the medicament container carrier is external of the housing, or at least arranged distally of its final proximal position within the housing; and in an assembled state in which the medicament container carrier is arranged in its final proximal position within the housing, which is further described with reference to the second aspect of the invention.

There is according to a second aspect of the present disclosure provided a method of assembly of a medicament delivery device comprising a sub-assembly according to the first aspect of the invention, or a method of assembly of a sub-assembly according to the first aspect of the invention. The method comprises: providing the retaining element in the housing, the retaining element being releasably engaged to the distally extending protrusion of the cap by means of the first and second engaging structures; inserting a medicament container in the medicament container carrier to provide a medicament container sub-assembly, the medicament container comprising a delivery member for expelling a medicament and a shield covering the delivery member; inserting the medicament container sub-assembly into the housing such that the medicament container is arranged in a final proximal position within the housing, wherein the retaining element is locked to the open proximal end of the carrier wall by means of the first and second locking structures, and wherein the inner surface of the retaining element is arranged adjacent to the first and second leg portions of the carrier wall to radially support the first and second leg portions to prevent the first and second leg portions from moving radially outwards. The distal end of the housing may be open. This can allow axial insertion of components such as a medicament container carrier, a retaining element and/or a medicament container into the distal end of the housing. The distal end of the housing may subsequently be closed by attaching a rear cap to the distal end of the housing.

Effects and features of the second aspect of the invention are largely analogous to those described above in connection with the first aspect of the invention. Embodiments mentioned in relation to the first aspect of the invention are largely compatible with the second aspect of the invention, of which some are exemplified below.

Thus, by the first and second locking structures, and the first and second engaging structures, the retaining element is efficiently provided in its correct position within the housing to radially support the first and second leg portions.

According to one embodiment, the step of providing the retaining element in the housing comprises installing the retaining element in its correct position within the housing by means of the cap as the retaining element is releasably engaged to the distally extending protrusion of the cap. Thus, the retaining element may be moved inside the housing by means of moving the cap. Subsequently, as the medicament container carrier, or medicament container sub-assembly, is inserted into the housing, the retaining element will be locked in its final position within the housing by locking to the medicament container carrier by means of the first and second locking structure, as previously described.

According to one embodiment, the carrier wall comprises a proximal supporting collar arranged around an inner periphery of the carrier wall, wherein the step of inserting the medicament container in the medicament container carrier is performed such that the shield passes the open proximal end and a neck of the medicament container is supported by the proximal supporting collar.

Thus, during assembly, the medicament container is inserted into the medicament container carrier such that a shield (e.g. RNS) attached to the medicament container passes the proximal supporting collar. In more detail, the shield is able to pass the proximal supporting collar as the first and second leg portions of the proximal open end of the carrier wall is moved from a resting position to a flexed position by that the first and second leg portions are moved or flexed outwardly by the shield upon a proximally applied force from the medicament container during its insertion into the medicament container carrier. After the shield has passed the proximal supporting collar, the first and second leg portions are moved, or flexed, inwardly back to its resting position to enable the proximal supporting collar to support a neck of the medicament container. The medicament container sub-assembly may then be inserted into the housing from the distal end until the final proximal position of the medicament container (and the medicament container carrier) is reached. In the final proximal position of the medicament container, the inner surface of the retaining element is arranged adjacent to the first and second leg portions of the carrier wall to radially support the first and second leg portions to prevent the first and second leg portions from moving radially outwards, as previously described.

The medicament delivery device, or sub-assembly, may thereafter be activated by removing the cap and the distally extending protrusion from the proximal end of the housing such that the retaining element is released from the distally extending protrusion by the release of the first engaging structure from the second engaging structure.

There is according to a third aspect of the present disclosure provided a kit of parts comprising an external fixture and a sub-assembly for a medicament delivery device configured to expel medicament from a medicament container. The sub-assembly comprises: a housing having a proximal end, a distal end and a window arranged between the proximal and distal ends; and a medicament container carrier extending along a longitudinal axis from a proximal end to a distal end. The medicament container carrier comprises: a carrier wall having an open proximal end, at least one slot extending from the open proximal end in a distal direction, and at least a first leg portion and a second leg portion radially separated by the slot at least at the open proximal end such that the first and second leg portions are radially movable relative each other. The sub-assembly further comprises a tubular shape retaining element extending along a longitudinal axis from a proximal end to a distal end, the retaining element comprising a proximal end portion extending distally from the proximal end, a distal end portion extending proximally of the distal end, and inner surface arranged adjacent to the first and second leg portions to radially support the first and second leg portions, wherein the distal end portion of the retaining element comprises a protrusion extending radially outwards from an outer periphery of the distal end portion. The external fixture is configured to move the retaining element into its final position within the housing.

Effects and features of the third aspect of the present disclosure are largely analogous to those described above in connection with the first aspect of the present disclosure. However, for the third aspect of the present disclosure, the retaining element is installed into its correct position within the housing by means of the external fixture, and not by means of the cap and the first and second engaging structures as described with reference to the first and second aspects of the present disclosure. Embodiments mentioned in relation to the first aspect of the present disclosure, at least concerning the housing and the medicament container carrier are largely compatible with the third aspect of the present disclosure.

According to one embodiment, the external fixture comprises an engagement arm configured to extend into the window of the housing and engage with the protrusion of the retaining element for moving the retaining element into its final position within the housing in which the protrusion engages with an associated recess of an inner surface of the housing, such that the retaining element is locked in position in the housing by means of the protrusion and the recess. The final position of the retaining element within the housing is the same as the previously mentioned correct position of the retaining element within the housing.

There is according to a fourth aspect of the present disclosure provided a method of assembly of a medicament delivery device (or a method of assembly of a sub-assembly of a medicament delivery device) using the kit of parts according to the third aspect of the present disclosure. The method comprises: moving the retaining element into its final position in the housing by means of the external fixture so that the engagement arm engages with the protrusion of the retaining element via the window of the housing; locking the retaining element in its final position within the housing by engaging the protrusion with the associated recess of the inner surface of the housing; inserting a medicament container in the medicament container carrier to provide a medicament container sub-assembly, the medicament container comprising a delivery member for expelling a medicament and a shield covering the delivery member; inserting the medicament container sub-assembly into the housing such that the medicament container is arranged in its final proximal position within the housing, wherein the inner surface of the retaining element is arranged adjacent to the first and second leg portions of the carrier wall to radially support the first and second leg portions to prevent the first and second leg portions from moving radially outwards.

Effects and features of the fourth aspect of the present disclosure are largely analogous to those described above in connection with the third aspect of the present disclosure. Embodiments mentioned in relation to the third aspect of the present disclosure are largely compatible with the fourth aspect of the present disclosure, of which some are exemplified below.

According to one embodiment, the method comprises: engaging the external fixture with the housing by inserting the engagement arm into the window of the housing and engaging with the protrusion of the retaining element; subsequent to locking the retaining element in its final position within the housing by engaging the protrusion with the associated recess of the inner surface of the housing, removing the external fixture from the housing by withdrawing the engagement arm through the window.

According to one embodiment, the carrier wall comprises a proximal supporting collar arranged around an inner periphery of the carrier wall, wherein the step of inserting the medicament container in the medicament container carrier is performed such that the shield passes the open proximal end and a neck of the medicament container is supported by the proximal supporting collar, as described in the corresponding step in the second aspect of the present disclosure.

There is according to a fifth aspect of the present disclosure provided a medicament delivery device for expelling medicament from a medicament container via a delivery member, the medicament delivery device comprising a sub-assembly according to the first aspect of the present disclosure.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

FIG. 1 schematically shows a perspective view of an example of a medicament delivery device;

FIG. 2 shows an exploded view of a sub-assembly of the medicament delivery device in FIG. 1;

FIG. 3 is a detailed perspective view of a retaining element of the sub-assembly of FIG. 2;

FIG. 4 is a detailed perspective view of a medicament container carrier of the sub-assembly of FIG. 2;

FIG. 5 is a detailed perspective view of the releasably engagement between the distally extending protrusion of the cap and the retaining element of the sub-assembly of FIG. 2;

FIG. 6A is a perspective view of a medicament container and the medicament container carrier of the sub-assembly of FIG. 2;

FIG. 6B is a perspective view of the medicament container inserted into the medicament container carrier of FIG. 6A for providing a medicament container sub-assembly;

FIG. 6C is a sectional view of the medicament container sub-assembly of FIG. 6B;

FIG. 6D is a sectional view of the medicament container inserted into the sub-assembly of FIG. 2;

FIG. 7 schematically shows a perspective view of an example external fixture and a medicament delivery device;

FIG. 8 shows an exploded view of a sub-assembly of the medicament delivery device in FIG. 7;

FIG. 9A is a detailed perspective view of a retaining element of the sub-assembly of FIG. 8;

FIG. 9B is a detailed perspective view of the external fixture of FIG. 7;

FIG. 10 is a perspective cross-sectional view of a medicament container inserted into the sub-assembly of FIG. 8 using the external fixture of FIG. 9B; and

FIGS. 11A-11D shows a method of assembly of the medicament delivery device of FIG. 7 comprising the sub-assembly of FIG. 8 using the external fixture 160 of FIG. 9B.

FIG. 12 shows a perspective view of the medicament container carrier of FIG. 4.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

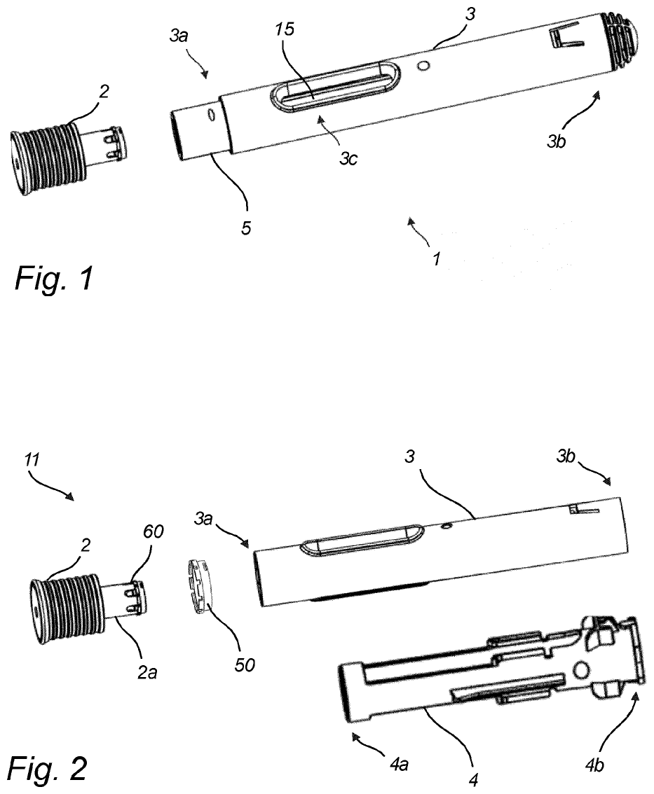

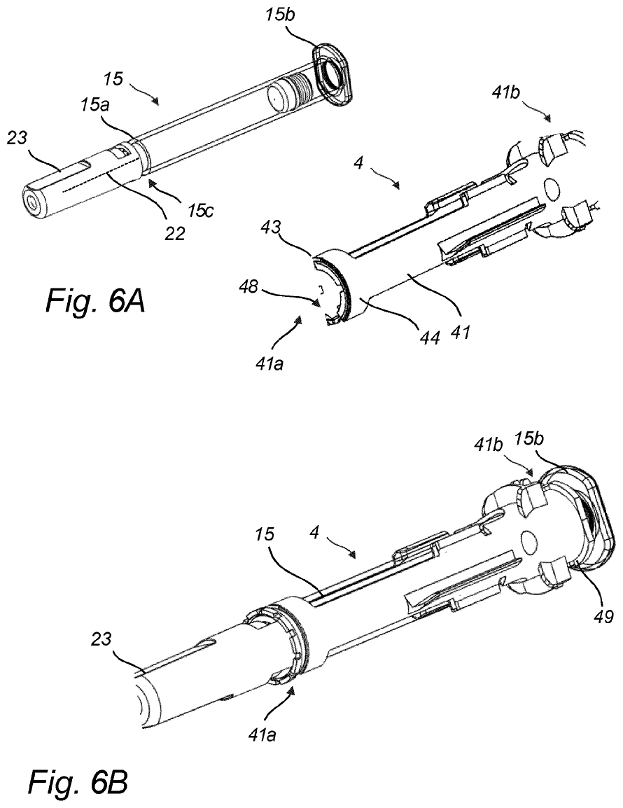

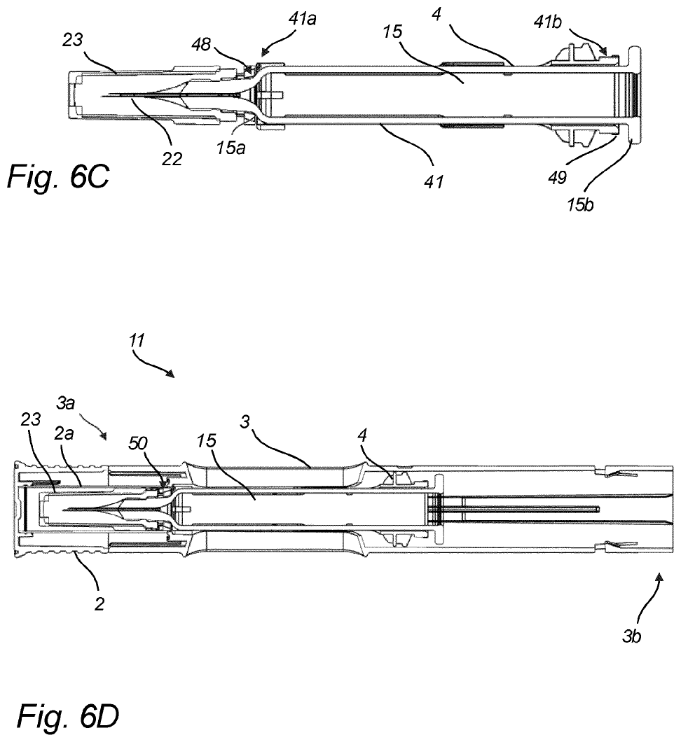

FIG. 1 shows an example of a medicament delivery device 1 configured to expel medicament from a medicament container 15 loaded in a housing 3 of the medicament delivery device 1. The housing 3 has a proximal end 3a and a distal end 3b, and a window 3c, or opening, arranged between the proximal end 3a the distal end 3b for visual inspection of the medicament container 15. The housing 3 may comprise a second window (not shown) arranged on an opposite side of the housing as compared to the window 3c. The medicament delivery device 1 may e.g. be an autoinjector or pen injector. In the embodiment of FIG. 1, the medicament delivery device 1 comprises a delivery member cover 5 arranged to cover a delivery member, typically a needle, of the medicament container 15 prior to usage of the medicament delivery device 1. The delivery member cover 5 is arranged in the housing 3 and extends proximally from the proximal end 3a of the housing 3. The medicament delivery device 1 may e.g. be triggered to discharge medicament from the medicament container 15 by movement of the delivery member cover 5 further into the housing 3 resulting in an exposure of the delivery member. The medicament delivery device 1 further comprises a cap 2 removably arranged to the housing 3 or the delivery member cover 5. The cap 2 is arranged at least partly proximal the housing 3. The distal end 3b of the housing is open as shown in FIG. 6D, for example, and the open distal end can be covered by a rear cap 19 (see FIG. 1), which can be attached to the housing once components have been inserted into the housing (such as the medicament container carrier 4, the retaining element 50 and the medicament container 15)

FIG. 2 shows an exploded view of a sub-assembly 11 of the medicament delivery device 1 of FIG. 1. The sub-assembly 11 comprises the housing 3 with its proximal end 3a and distal end 3b as described with reference to FIG. 1, and a medicament container carrier 4 extending along a longitudinal axis from a proximal end 4a to a distal end 4b. The medicament container carrier 4 may, after assembly, be fixedly attached to the housing 3 of the medicament delivery device 1, e.g. by a snap lock or other conventional locking means. The medicament container carrier 4 and the housing 3 are preferably made of the same material, e.g. plastic. The medicament container carrier 4 is configured to house and support the medicament container 15 (shown in FIG. 1).

The sub-assembly 11 further comprises the cap 2 described with reference to FIG. 1. The cap 2 comprises a protrusion 2a arranged to extend distally into the proximal end 3a of the housing 3. The distally extending protrusion 2a is typically tubular.

The sub-assembly 11 further comprises a tubularly shaped retaining element 50 arranged inside the housing 3 and configured for interacting with the medicament container carrier 4 as will be described in the following.

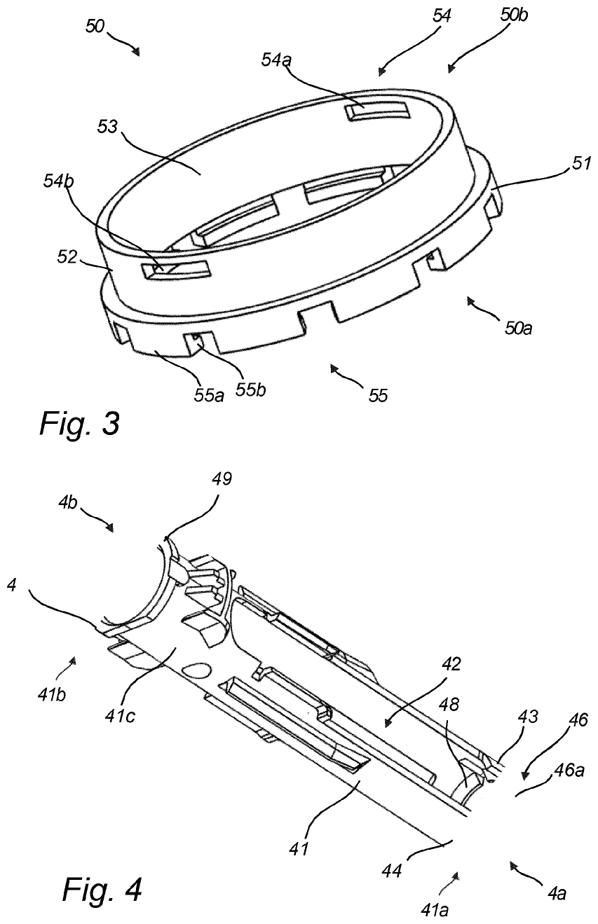

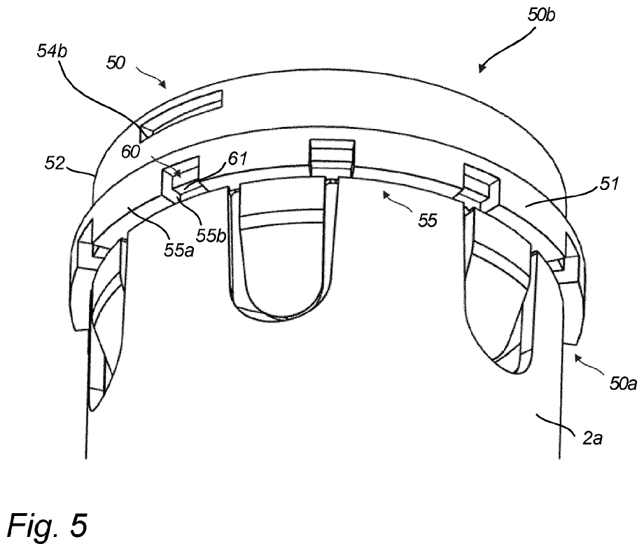

The tubularly shaped retaining element 50 is shown in greater detail in FIG. 3 and FIG. 5. The retaining element 50 extends along a longitudinal axis from a proximal end 50a to a distal end 50b. The retaining element 50 comprises a proximal end portion 51 extending distally from the proximal end 50a, and a distal end portion 52 extending proximally from the distal end 50b. For example, the distal end portion 52 extends up to 50%, such as e.g. up to 25% of the longitudinal extension of the retaining element 50, and the proximal end portion 51 extends up to 50%, such as e.g. up to 25% of the longitudinal extension of the retaining element 50.

The retaining element 50 comprises an inner surface 53, e.g. being an inner surface of the distal end portion 52. The distal end portion 52 comprises a first locking structure 54 which in FIG. 3 is embodied as two oppositely arranged locking recesses 54a, 54b. The locking recesses 54a, 54b may be through holes, or recesses in the inner surface 53. The retaining element 50 further comprises a first engaging structure 55, which in FIG. 3 is embodied as at least one flexible arm 55a having a hooked-shaped end 55b.

The medicament container carrier 4 is shown in greater detail in FIG. 4. The medicament container carrier 4 comprises a carrier wall 41 having an open proximal end 41a, and an open distal end 41b. The open proximal end 41a is arranged at the proximal end 4a of the medicament container carrier 4, and the open distal end 41b is arranged in a distal end portion 41c of the carrier wall 41, at the distal end 4b of the medicament container carrier 4.

The medicament container carrier 4 comprises a distal supporting collar 49 at the open distal end 41b. The distal supporting collar 49 is arranged around an outer periphery of the carrier wall 41. The distal supporting collar 49 is configured to support a flange of the medicament container 15, as will be described with reference to FIGS. 6A-6D.

The carrier wall 41 comprises a proximal supporting collar 48 arranged around an inner periphery of the carrier wall 41 at the open proximal end 41a. The proximal supporting collar 48 is configured to support a neck of the medicament container 15, as will be described with reference to FIGS. 6A-6D.

The medicament container carrier 4 comprises a slot 42 extending from the open proximal end 41a in a distal direction to the distal end portion 41c. The slot 42 ends in the distal end portion 41c prior the open distal end 41b. As a result of the slot 42, the carrier wall 41 is divided into a first leg portion 43 and a second leg portion 44 radially separated by the slot 42 at the open proximal end 41a. Thus, the first and second leg portions 43, 44 are radially movable relative each other. The medicament container carrier 4 comprises a second locking structure 46 extending from at least one of the first and second leg portions 43, 44 of the carrier wall 41. In the embodiment shown in FIG. 4, the locking structure 46 comprises at least one locking protrusion 46a. The locking protrusions can also be seen in FIG. 12. Optionally, as shown in FIG. 12, locking protrusions can additionally (or alternatively) be provided elsewhere on the proximal end of the medicament container carrier 4.

The medicament container carrier 4 is lockable with the retaining element 50 such that in an assembled state, at least one of the first locking recesses 54a, 54b is snap-locked with the locking protrusion 46a. Hereby the retaining element 50 is locked to the open proximal end 41a of the carrier wall 41, and thus, the inner surface 53 is arranged adjacent to the first and second leg portions 43, 44 to radially support the first and second leg portions 43, 44.

The distal end of the distally extending protrusion 2a of the cap 2 is shown together with the retaining element 50 in greater detail in FIG. 5. The distally extending protrusion 2a comprises a second engaging structure 60, here embodied as a circumferential protrusion 61. The first engaging structure 55, i.e. in FIG. 5 the flexible arm 55a with the hooked-shaped end 55b, is releasably engageable with the second engaging structure 60, i.e. in FIG. 5 the circumferential protrusion 61. Thus, the retaining element 50 is releasably engaged with the distally extending protrusion 2a.

Turning to FIGS. 6A-6D showing components of the sub-assembly 11 of FIG. 2 in various states together with a medicament container 15. In FIG. 6A, the medicament container 15 is shown in its unassembled state relative to the medicament container carrier 4.

In FIG. 6A, the medicament container 15 is shown together with a shield 23, e.g. a rigid needle shield, RNS, protecting the delivery member 22 (schematically shown as a dashed line in FIG. 6A) of the medicament container 15. The shield 23 is removably arranged relative to the body of the medicament container 15, and is arranged proximally a neck 15a of the medicament container 15. The neck 15a is separated from the shield 23 by a portion 15c of the medicament container 15 having a smaller diameter compared to the neck 15a and the shield 23. The shield 23 and the neck 15a may have the same, or substantially the same, diameter. Moreover, the medicament container 15 comprises a collar 15b arranged at the distal end of the medicament container 15.

During assembly, the medicament container 15 is inserted into medicament container carrier 4 via the open distal end 41b of the carrier wall 41. During the insertion, the shield 23 of the medicament container 15 passes the proximal supporting collar 48 via the open proximal end 41a. The shield 23 is able to pass the proximal supporting collar 48 and through the open proximal end 41a as the first and second leg portions 43, 44 are moved, or flexed, outwardly as the medicament container 15 moves proximally and the shield 23 pushes the first and second leg portions 43, 44 apart. In this respect, the diameter of the opening of the proximal open end 41a (prior to receiving the shield 23) is smaller than the diameter of the shield 23. After the shield 23 has passed the proximal supporting collar 48 and through the open proximal end 41a, the first and second leg portions are moved, or flexed, inwardly back to their initial position. The first and second leg portions 43, 44 are able to flex inwardly back to their initial position owing to the smaller diameter of the portion 15c of the medicament container 15. Hereby, the medicament container 15 is inserted into the medicament container carrier 4 to provide a medicament container sub-assembly, as is shown in FIG. 6B.

FIG. 6C shows a cross sectional view of the medicament container sub-assembly of FIG. 6B along the longitudinal direction. Here, it is clearly shown that the proximal supporting collar 48 supports the neck 15a of the medicament container 15. In FIGS. 6B-6C, the medicament container 15 is arranged in a final proximal position within the medicament container carrier 4. Moreover, the distal supporting collar 49 is configured to support the flange 15b of the distal end of the medicament container 15. As shown in FIG. 6C, there may be a small play between the flange 15b and the distal supporting collar 49.

The medicament container sub-assembly of FIGS. 6B-6C may then be inserted into the housing 3 from the distal end 3b until a final proximal position of the medicament container carrier 4 is reached within the housing 3, which is shown in FIG. 6D. Here, the cap 2 with is distally extending protrusion 2a is engaged to the housing 3, such that the retaining element 50 is arranged in its correct position within the housing 3. In the final proximal position of the medicament container carrier 4 within the housing 3, the medicament container carrier 4 is locked to the retaining element 50 by at least one of the first locking recesses 54a, 54b being snap-locked with the locking protrusion 46a. Hereby the retaining element 50 is locked to the open proximal end 41a of the carrier wall 41, and thus, the inner surface 53 is arranged adjacent to the first and second leg portions 43, 44 to radially support the first and second leg portions 43, 44 and to prevent the first and second leg portions 43, 44 from moving radially outwards.

A method of assembly of a medicament delivery device comprising a sub-assembly 11 as shown in FIGS. 6A-6D may be described as follows. In a first step S1, the retaining element 50 is provided in the housing 3, the retaining element 50 is releasably engaged to the distally extending protrusion 2a of the cap 2 by means of the first and second engaging structures 55, 60.

In a second step S2, a medicament container 15 is inserted in the medicament container carrier 4 to provide a medicament container sub-assembly, the medicament container 15 comprises a delivery member 22 for expelling a medicament and a shield 23 covering the delivery member 23, as shown in FIG. 6A. In a third step S3, the medicament container sub-assembly is inserted into the housing 3 such that the medicament container 15, and the medicament container carrier 4, are arranged in their respective final proximal position within the housing 3, wherein the retaining element 50 is locked to the open proximal end 41a of the carrier wall 41 by means of the first and second locking structures 54, 46. In this position of the retaining element 50, the inner surface 53 of the retaining element 50 is arranged adjacent to the first and second leg portions 43, 44 of the carrier wall 41 to radially support the first and second leg portions 43, 44. Hereby, the first and second leg portions 43, 44 are prevented from moving radially outwards.

The first step S1 of providing the retaining element 50 in the housing 3 may comprise installing the retaining element 50 in its correct position within the housing 3 by means of the cap 2 as the retaining element 50 is releasably engaged to the distally extending protrusion 2a of the cap 2. Thus, the retaining element 50 may be moved inside the housing 3 by means of moving the cap 2. The second step S2 of inserting the medicament container 15 in the medicament container carrier 4 is typically performed such that the shield 23 passes the open proximal end 41a and the neck 15a of the medicament container 15 is supported by the proximal supporting collar 48, as described with reference to FIGS. 6B-6C. In the third step S3, the medicament container carrier 4 in the medicament container sub-assembly is inserted into the housing 3 such that the retaining element 50 is locked in its final position within the housing 3 by locking to the medicament container carrier 4 by means of the first and second locking structure 54, 46, as previously described.

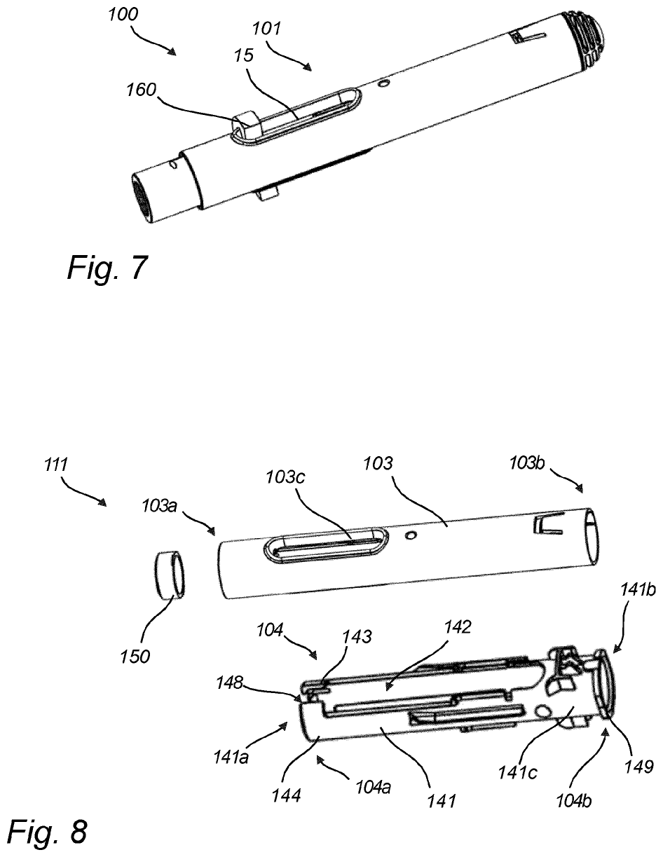

FIG. 7 shows a kit of parts 100 comprising an external fixture 160 and a sub-assembly 111 (shown in FIG. 8) for a medicament delivery device 101 configured to expel medicament from a medicament container 15. The medicament container 15 is for example the same as that described with reference to FIG. 6A.

FIG. 8 shows an exploded view of a sub-assembly 111 of the medicament delivery device 1 of FIG. 7. The sub-assembly 111 comprises the housing 103 having a proximal end 103a, and distal end 103 and a window 103c arranged between the proximal and distal ends 103a, 103b. The window 103c is typically a first window 103c as the housing 103 may comprise a second window (not shown) arranged on an opposite side of the housing 103 as compared to the first window 103c.

The sub-assembly 111 further comprises a medicament container carrier 104 extending along a longitudinal axis from a proximal end 104a to a distal end 104b. The medicament container carrier 104 may, after assembly, be fixedly attached to the housing 103 of the medicament delivery device 101, e.g. by a snap lock or other conventional locking means. The medicament container carrier 104 and the housing 103 are preferably made of the same material, e.g. plastic. The medicament container carrier 104 is configured to house and support the medicament container 15 (shown in FIG. 7).

The medicament container carrier 104 comprises a carrier wall 141 having an open proximal end 141a, and an open distal end 141b. The open proximal end 141a is arranged at the proximal end 104a of the medicament container carrier 104, and the open distal end 141b is arranged in a distal end portion 141c of the carrier wall 141, at the distal end 104b of the medicament container carrier 104.

The medicament container carrier 104 comprises a distal supporting collar 149 at the open distal end 141b. The distal supporting collar 419 is arranged around an outer periphery of the carrier wall 141. The distal supporting collar 149 is configured to support a flange of the medicament container 15 in a corresponding manner as described with reference to the medicament container carrier 4 of FIG. 4. The carrier wall 141 comprises a proximal supporting collar 148 arranged around an inner periphery of the carrier wall 141 at the open proximal end 141a. The proximal supporting collar 148 is configured to support a neck of the medicament container 15, in a corresponding manner as described with reference to the medicament container carrier 4 of FIG. 4.

The medicament container carrier 104 comprises a slot 142 extending from the open proximal end 141a in a distal direction to the distal end portion 141c. The slot 142 ends in the distal end portion 141c prior the open distal end 141b. As a result of the slot 142, the carrier wall 141 is divided into a first leg portion 143 and a second leg portion 144 radially separated by the slot 142 at the open proximal end 141a. Thus, the first and second leg portions 143, 144 are radially movable relative each other.

The sub-assembly 111 further comprises a tubularly shaped retaining element 150 arranged inside the housing 103 and configured for interacting with the medicament container carrier 104 as will be described in the following.

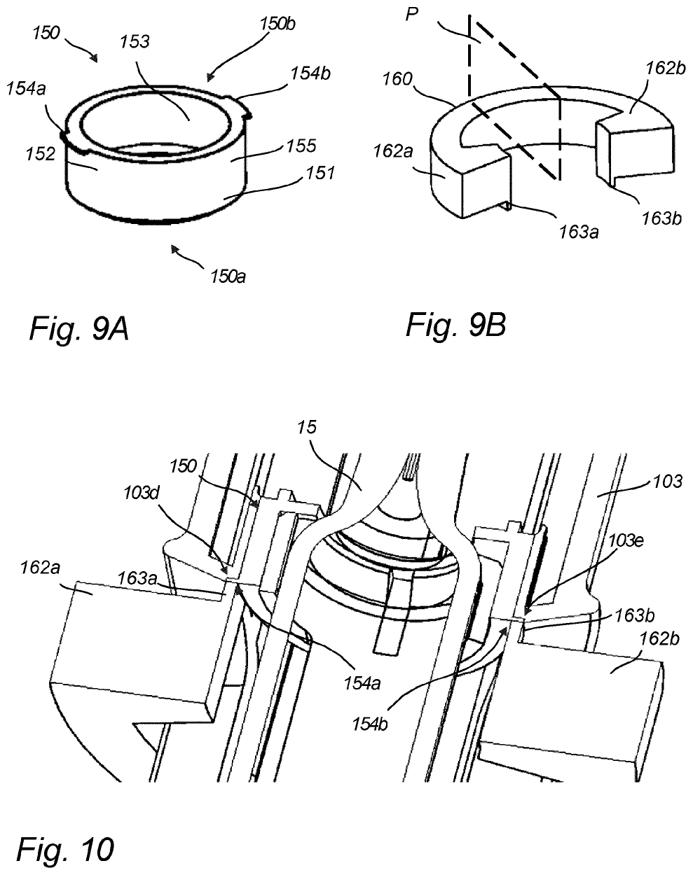

The tubularly shaped retaining element 150 is shown in greater detail in FIG. 9A. The retaining element 150 extends along a longitudinal axis from a proximal end 150a to a distal end 150b. The retaining element 150 comprises a proximal end portion 151 extending distally from the proximal end 150a, and a distal end portion 152 extending proximally from the distal end 150b. For example, the distal end portion 152 extends up to 50%, such as e.g. up to 25% of the longitudinal extension of the retaining element 150, and the proximal end portion 151 extends up to 50%, such as e.g. up to 25% of the longitudinal extension of the retaining element 150.

The retaining element 150 comprises an inner surface 153, e.g. being an inner surface of the distal end portion 152. The retaining element 150 further comprises at least one protrusion 154, here embodied as a first protrusion 154a and a second protrusion 154b, extending radially outwards from an outer surface 155 of the distal end portion 152.

The external fixture 160 is shown in greater detail in FIG. 9B. The external fixture comprises at least one engagement arm 162, here embodied as a first engagement arm 162a and a second engagement arm 162b arranged symmetrically relative a centre plane P. The first and second engagement arms 162, 162b are configured to extend into the housing 103 via first and second windows 103c on opposite sides of the housing 103. Each one of the first and second engagement arms 162a, 162b comprises a lip 163a, 163b. The first and second engagement arms 162a, 162b are configured to engage with the first and second protrusions 154a, 154b of the retaining element 150 by means of the lips 163a, 163b. Hereby, the external fixture 160 may move the retaining element 150 into its final position within the housing 103 as will be described in the following.

As shown in FIG. 10, in the final position of the retaining element 150, the first and second protrusions 154a, 154b engages with an associated recess 103d, 103e of an inner surface of the housing 103. Hereby, the retaining element 150 is locked in position in the housing 103 by means of the protrusions 154a, 154b and the recesses 103d, 103e using the external fixture 160.

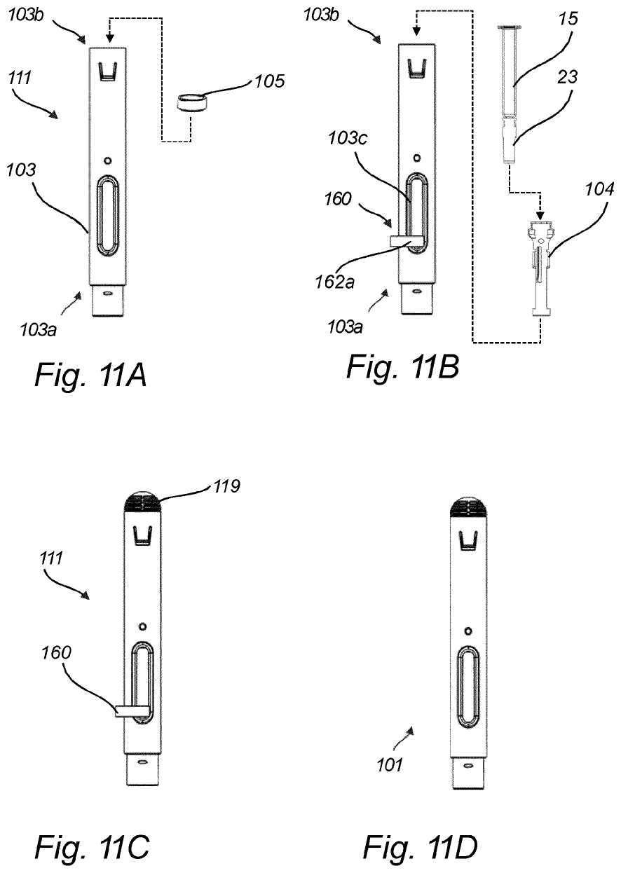

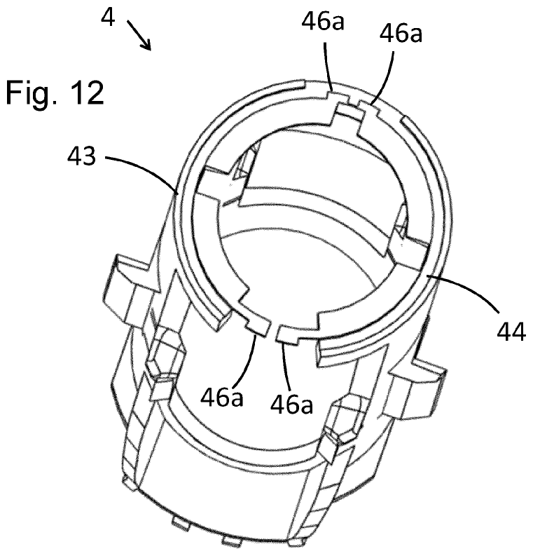

A method of assembly of a medicament delivery device 101 comprising a sub-assembly 111 as shown in FIG. 8 and using an external fixture 160 as shown in FIG. 9B will now be described with reference to FIGS. 11A, 11B, 11C, 11D. In a first step S101, shown in FIG. 11A, the sub-assembly 111 is provided in which the retaining element 150 has been inserted into the housing 103 via the distal end 103b. Typically, the retaining element 150 is moved within the housing 103 as far to the proximal end 103a as possible.

In a second step S102, shown in FIG. 11B, the retaining element 150 is moved into its final position in the housing 103 by means of the external fixture 160. The external fixture 160 is thereby pressed in the proximal direction (for example by the machinery that is assembling the device or by the person assembling the device), e.g. with a force to overcome any counteracting force (owing to e.g. internal coil springs or the like). In the second step S102, the external fixture 160 engages with the housing 103 by insertion of the engagement arms 162a, 162b (of which only the first engagement arm 162a is visible in FIG. 11B) into the windows of the housing 103 (of which only the first window 103c is visible in FIG. 11B). Hereby, the engagement arms 162a, 162b and the corresponding lips engage with the first and second protrusions of the retaining element 150 as previously described. The external fixture is typically attached to the housing only after the retaining element is inserted into the housing, although the external fixture could initially be attached to the housing before (or while) the retaining element is inserted into the housing; this could be achieved by a sloping proximal end of the retaining element and/or a sloping distal end of the external fixture so that the retaining element can push the engagement arms 162a, 162b of the external fixture apart to pass the external fixture.

In a third step S103, the retaining element 150 is locked in its final position within the housing 103 using the external fixture 160 by engaging the first and second protrusions of the retaining element 150 with the associated first and second recesses of the inner surface of the housing 103, as previously described. In one example, the external fixture is held in place relative to the housing (and therefore the retaining element is locked in place relative to the housing) by biasing the external fixture so that the engagement arms 162a, 162b are stretched apart when the external fixture engages the housing, resulting in a friction lock that can be released in the seventh step below by pulling the engagement arms 162a, 162b apart. Alternatively or additionally, other ways of holding the external fixture in place relative to the housing (therefore locking the retaining element in place relative to the housing) could also be used, including a releasable snap fit between the housing and the external fixture.

In a fourth step S104, medicament container 15 is inserted in the medicament container carrier 104 to provide a medicament container sub-assembly, as shown in FIG. 11B. The medicament container 15 comprising a delivery member for expelling a medicament and a shield 23 covering the delivery member as previously described. As previously described, the carrier wall comprises a proximal supporting collar arranged around an inner periphery of the carrier wall, wherein the step S104 of inserting the medicament container 15 in the medicament container carrier 104 is performed such that the shield 23 passes the open proximal end and a neck of the medicament container 15 is supported by the proximal supporting collar.

In a fifth step S105, the medicament container sub-assembly is inserted into the housing 103 via the distal end 103b, such that the medicament container 15, and the medicament container carrier 140, are arranged in their respective final proximal position within the housing 103. Hereby, the inner surface of the retaining element 150 is arranged adjacent to the first and second leg portions of the carrier wall to radially support the first and second leg portions. Hereby, the first and second leg portions are prevented from moving radially outwards.

In a sixth step S106, a rear cap 119 is closing the distal end 103b of the housing 103, as shown in FIG. 11C.

In a seventh step S107, the external fixture 160 is removed from the housing 103 by withdrawing the engagement arms 162a, 162b through the windows of the housing 103 to provide a medicament delivery device 101, as shown in FIG. 11D.

The inventive concept has mainly been described above with reference to a few examples. However, as is readily appreciated by a person skilled in the art, other embodiments than the ones disclosed above are equally possible within the scope of the inventive concept, as defined by the appended claims. For example, the drive member could for example be motor driven or manually driven instead of having an automatic spring-loaded structure.

1-15. (canceled)

16. A sub-assembly for a medicament delivery device configured to expel medicament from a medicament container, the sub-assembly comprising:
a housing having a proximal end and a distal end; and
a medicament container carrier extending along a longitudinal axis from a proximal end to a distal end, the medicament container carrier comprising:
a carrier wall having an open proximal end, at least one slot extending from the open proximal end in a distal direction, and at least a first leg portion and a second leg portion radially separated by the at least at the open proximal end such that the first and second leg portions are radially movable relative to each other;
a tubularly shaped retaining element extending along a longitudinal axis from a proximal end to a distal end, the retaining element comprising a proximal end portion extending distally from the proximal end, a distal end portion extending proximally from the distal end, and an inner surface adjacent to the first and second leg portions to radially support the first and second leg portions; and
a cap arranged at least partly proximal the housing, the cap comprising a protrusion extending distally into the proximal end of the housing,
wherein the distal end portion of the retaining element comprises a first locking structure lockable with an associated second locking structure extending from at least one of the first and second leg portions of the carrier wall, such that the retaining element is locked to the open proximal end of the carrier wall by means of the first and second locking structures, and wherein the proximal end portion of the retaining element comprises a first engaging structure releasably engageable with an associated second engaging structure of the distally extending protrusion, such that the retaining element is releasably engaged to the distally extending protrusion.

17. The sub-assembly according to claim 16, being configured to move from a pre-activation preventing state in which the retaining element is releasably engaged to the distally extending protrusion, into an activation admittible state in which the cap is removed and the distally extending protrusion is released from the retaining element by the release of the first engaging structure from the second engaging structure.

18. The sub-assembly according to claim 16, wherein the first and second locking structures are configured as a snap-lock.

19. The sub-assembly according to claim 18, wherein the snap-lock comprises a recess and a protrusion configured to be snap-locked into the recess, wherein the first locking structure comprise the recess or the protrusion, and the second locking structure comprises the other one of the recess and the protrusion.

20. The sub-assembly according to claim 18, wherein the first engaging structure comprises at least one flexible arm having a hooked-shaped end, and the second engaging structure comprises a protrusion configured to engage with the hook-shaped end of the flexible arm in a releasably engaging manner.

21. The sub-assembly according to claim 20, wherein the protrusion of the second engaging structure is a circumferential protrusion.

22. The sub-assembly according to claim 20, wherein the first engaging structure comprises a plurality of flexible arms arranged around the periphery of the retaining element, each one of the flexible arms having a hooked-shaped end engageable with the protrusion of the second engaging structure.

23. The sub-assembly according to claim 16, wherein the locking between the retaining element and the open proximal end of the carrier wall by means of the first and second locking structures, are stronger than the releasable engagement of the retaining element and the distally extending protrusion of the cap by means of the first and second engaging structures.

24. The sub-assembly according to claim 23, wherein the first and second engaging structures are configured to be released from each other upon an applied threshold force to the cap in the proximal direction, wherein the first and second locking structures are configured to remained locked to each other upon the application of the threshold force to the cap in the proximal direction.

25. A method of assembly of the sub-assembly according to claim 16, wherein the method comprises:
providing the retaining element in the housing, the retaining element being releasably engaged to the distally extending protrusion of the cap by means of the first and second engaging structures;
inserting a medicament container in the medicament container carrier to provide a medicament container sub-assembly, the medicament container comprising a delivery member for expelling a medicament and a shield covering the delivery member;
inserting the medicament container sub-assembly into the housing such that the medicament container is arranged in a final proximal position within the housing, wherein the retaining element is locked to the open proximal end of the carrier wall by means of the first and second locking structures, and wherein the inner surface of the retaining element is arranged adjacent to the first and second leg portions of the carrier wall to radially support the first and second leg portions to prevent the first and second leg portions from moving radially outwards.

26. The method according to claim 25, wherein the carrier wall comprises a proximal supporting collar arranged around an inner periphery of the carrier wall, and wherein the step of inserting the medicament container in the medicament container carrier is performed such that the shield passes the open proximal end and a neck of the medicament container is supported by the proximal supporting collar.

27. A kit of parts comprising an external fixture and a sub-assembly of a medicament delivery device configured to expel medicament from a medicament container, the sub-assembly comprising:
a housing having a proximal end, a distal end and a window arranged between the proximal and distal ends;
a medicament container carrier extending along a longitudinal axis from a proximal end to a distal end, the medicament container carrier comprising:
a carrier wall having an open proximal end, at least one slot extending from the open proximal end in a distal direction, and at least a first leg portion and a second leg portion radially separated by the slot at least at the open proximal end such that the first and second leg portions are radially movable relative each other; and
a tubular shape retaining element extending along a longitudinal axis from a proximal end to a distal end, the retaining element comprising a proximal end portion extending distally from the proximal end, a distal end portion extending proximally of the distal end, and inner surface arranged adjacent to the first and second leg portions to radially support the first and second leg portions, wherein the distal end portion of the retaining element comprises a protrusion extending radially outwards from an outer surface of the distal end portion;
wherein the external fixture is configured to move the retaining element into its final position within the housing.

28. The kit of parts according to claim 27, wherein the external fixture comprises an engagement arm configured to extend into the window of the housing and engage with the protrusion of the retaining element for moving the retaining element into its final position within the housing in which the protrusion engages with an associated recess of an inner surface of the housing, such that the retaining element is locked in position in the housing by means of the protrusion and the recess.

29. A method of assembly of a sub-assembly of a medicament delivery device using the kit of parts according to claim 27, wherein the method comprises:
moving the retaining element into its final position in the housing by means of the external fixture so that the engagement arm engages with the protrusion of the retaining element via the window of the housing;
locking the retaining element in its final position within the housing by engaging the protrusion with the associated recess of the inner surface of the housing;
inserting a medicament container in the medicament container carrier to provide a medicament container sub-assembly, the medicament container comprising a delivery member for expelling a medicament and a shield covering the delivery member;
inserting the medicament container sub-assembly into the housing such that the medicament container is arranged in its final proximal position within the housing, wherein the inner surface of the retaining element is arranged adjacent to the first and second leg portions of the carrier wall to radially support the first and second leg portions to prevent the first and second leg portions from moving radially outwards.

30. The method according to claim 29, further comprising:
engaging the external fixture with the housing by inserting the engagement arm into the window of the housing and engaging with the protrusion of the retaining element; and
subsequent to locking the retaining element in its final position within the housing by engaging the protrusion with the associated recess of the inner surface of the housing, removing the external fixture from the housing by withdrawing the engagement arm through the window.
